SEQUENCE LISTING (4 sequences)

SEQ ID NO: 1 - protein, 8 aa, Homo Sapiens
RTNKEASI

SEQ ID NO: 2 - protein, 7 aa, Homo Sapiens
  VARIANT (4)...(0): Xaa at position 4 can be any amino acid, this sequence is the same as Seq ID N2 of WO A 001458
GSAXFTN

SEQ ID NO: 3 - protein, 6 aa, Homo Sapiens
NQRNRD

SEQ ID NO: 4 - protein, 9 aa, Homo Sapiens
GPPEVQNAN

(19) United States
(12) Patent Application Publication
(10) Pub. No.: US 2004/0022870 A1
(43) Pub. Date: Feb. 5, 2004

(54) Immunogenic TLP composition

(76) Inventor: Giulio Tarro, Napoli (IT)

Correspondence Address: BAKER & BOTTS, 30 ROCKEFELLER PLAZA, NEW YORK, NY, 10112

(21) Appl. No.: 10/631,838

(22) Filed: Jul. 31, 2003

Related U.S. Application Data
(63) Continuation of application No. 09/284,051, filed on Jun. 9, 1999.
Application No. 09/284,051 is a 371 of international application No. PCT/IT97/00240, filed on Oct. 9, 1997.

(30) Foreign Application Priority Data
Oct. 9, 1996 (IT) RM96A000687

(51) Int. Cl. A61K 38/08; A61K 31/7048; A61K 33/24

(52) U.S. Cl. 424/649; 514/15; 514/27

(57) ABSTRACT

The present invention relates to an immunogenic composition containing at least one protein from TLP or a fragment thereof, and in particular to the compositions wherein these fragments contain at least one of the peptides claimed as SEQ ID NO: 1, SEQ ID NO: 2, SEQ ID NO: 3 and SEQ ID NO: 4, suitable in therapy against tumoral diseases, and in particular against non-small cell lung carcinoma and urogenital cancer.

### CROSS REFERENCE TO RELATED APPLICATIONS

This is a United States national stage application based on International Application No. PCT/IT97/00240 filed Oct. 9, 1997 which claims priority to Italian Patent Application No. RM96A000687 filed Oct. 9, 1996.

### BACKGROUND OF THE INVENTION

The present invention relates to the field of immunotherapy of tumoral diseases.

### PRIOR ART

Oncology research has long been directing its efforts towards the problem of immunotherapy of tumoral diseases, on the basis of the reasonable possibility of finding a therapeutically useful solution through the manipulation of the immuno-oncolytic reaction that the human organism can develop spontaneously. The initial urge towards such a trend of scientific research can be recognized in the first observations (I. S. Irlin, Virology 1967;32:725; E. Klen et al., Natl. Cancer Inst. 1964;32:547; G. J. Pasternak, J. Nat. Cancer Inst. 1965;34:71; S. S. Tevethia et al., Immunol. 1968:100:358; R. Nishioka et al., Monograph 1968;7:49) regarding the stimulation of specific humoral and cellular antibodies by the antigens of neoplastic cells both in animals and in human beings.

The immune manipulations attempted so far as an immunotherapic approach, while reflecting the knowledge successively acquired concerning the pathophysiology of cancer and of the immune system of the host, on the other side also suffers from the lack of suitable immunogenic agents or from the difficulty experienced in removing the situations of block of cellular immunity which are present in the cancer patients.

As a matter of practice, two main roads are originally followed:

a) the non specific activation of the host's immunity in order to strengthen the immuno-oncolytic reactions (immuno-adjuvants);

b) the specific activation of the host's immunity in order to electively stimulate the production of antibodies having oncolytic effects.

The specific approach has the advantage to direct a more specific and effective immune response to the tumor cells, strengthening the effects.

In any case, the lysis of tumor cells by the immune system is mediated both by direct cytotoxic mechanisms (NK cells) and by cytotoxic mechanisms that are more complex but more effective and specific, involving recognition of antigens on the surface of the tumor cells and the presence of antibodies (ADCC cytotoxicity, prevalently mediated by CD8⁺ cells). The immune system always operates by means of a complex network of cytokines, which modulate the action of the effector cells by means of inhibition and stimulation, with a “cascade” mechanism.

Attempts at immunotherapy have to date been aimed at generic amplification of the cell-mediated immune response, either expanding (thymic hormones, IL-2) or activating (BCG, PPD, IFNs, IL-2, TNFs, etc.) the lymphocyte populations in a non-specific manner. Research into this effect was also through the use of a single substance (IL-2, IFN, etc.) which, in order to induce the necessary function, was required to be administered at high doses, with considerable toxic effects and prohibitively high costs (Mule J. J., Shu S., Swarz S. L., Rosenberg S. A., Science 1984;225:1478; Hadden J. W., in “Advances in immunomodulation”, 1988, Ed. B. Bizzini and E. Bonmassar, Pitagora Press Roma).

Previous studies have shown that combined immunotherapy (thymic hormones and a cytokine, IFN-alpha or IL-2, administered at a low dose) was capable of producing a synergistic effect on the cytotoxic activities of lymphocytes (NK, LAK, CTL activity) with respect to certain tumor cells in vitro (Favalli, C., Mastino, A., Jezzi, T., Grelli, S., Goldestein, A. L. and Garaci, E., Int. J. Immunopharmacol. 1989;11:443-450; Mastino, A., Favalli, C., Grelli, S., Innocenti, F. and Garaci, E., Cell. Immunol. 1991;133:196-205). However, an increase in cytotoxic activities corresponded neither to an adequate anti-tumor response in vivo, nor to an increased survival rate (Favalli, C., Mastino, A., Grelli, S., Pica, F., Rasi, G., Garaci, E., Combination Therapies. pp. 275-280, Ed. by A. Goldstein and E. Garaci, Plenum Press, N.Y., 1992; Mastino, A., Favalli, C., Grelli, S., Rasi, G., Pica, F., Goldestein, A. L. and Garaci, E., Int. J. Cancer. 1992;50:493-499; Garaci, E., Pica, F., Mastino, A., Palamara, A. T., Belardelli, F. and Favalli, C., J. Immunother. 1993;13:7-17). On the contrary, when combined immunotherapy was preceded by chemotherapy (even using ineffective doses) there was complete recovery from the tumor (Mastino, A., Favalli, C., Grelli, S., Rasi, G., Pica, F., Goldestein, A. L. and Garaci, E., Int. J. Cancer 1992;50:493-499; Garaci, E., Pica, F., Mastino, A., Palamara, A. T., Belardelli, F. and Favalli, C., J. Immunother. 1993;13:7-17; Rasi, G., Sinibaldi-Vallebona P., Favalli, C., Pierimarchi, P., et al., 2nd International Symposium on Combination Therapies, documents, 1-3 May, 1992 —Santa Tecla Conn.).

Further studies on experimental models have shown that the immunotherapy was only effective in case of immunogenic neoplasia, and that the main role of chemotherapy (used at ineffective doses) was to render the neoplasia immunogenic (Rasi, G., Sinibaldi-Vallebona P.,-Favalli, C., Pierimarchi, P., et al., 2nd International Symposium on Combination Therapies, documents, 1-3 May, 1992 —Santa Tecla Conn.; Sinibaldi-Vallebona P., Pierimarchi, P., Ravagnan, G. P., Rasi, G., Third International Symposium on Combination Therapies, documents, 29-31 Oct., 1993, Houston, Tex.; Sinibaldi-Vallebona P., Pierimarchi,. P., Lucertini, L., Ravagnan, G. P., Rasi, G., Fourth International Svmposium on Combination Therapies, 14-17 Jun., 1994, documents, p. 105; Rasi, G., Silecchia, G. F., Sinibaldi-Vallebona P., Pierimarchi, P., Sivilia, M., Tremiterra, S., Garaci, E., Int. J. Cancer 1994;57:701-705). Recently, this strategy has also been found to be effective in treatment of solid human tumors (Rasi, G., Favalli, C., Terzoli, E., Izzo, F., Sinibaldi-Vallebona P., Pierimarchi, P., Sivilia, M., Garaci, E., Biomedicine & Pharmacotherapy 1993;47:292). In experimental models, we have thus demonstrated the low effect or lack of effect of each of the single treatments (chemotherapy, thymic hormone, cytokine), the absence of anti-tumor effect even in the presence of a considerable increase in the levels of immune activity in cells, and lysis of the tumor only in the presence of a specific cell-modulated cytotoxic activity on neoplastic cells. From these studies it can therefore be stated that the role of the antigen is decisive in order to induce a cytotoxic immune response with a significant anti-tumor effect.

When attempting to amplify the anti-neoplastic immune response, the availability of antigens to induce and modulate, and the knowledge of the immunological relationships within each “target/effector” system (neoplastic cell/lymphocyte), therefore appear to be essential.

TLP complexes are protein complexes present in human tumor cells. Among these TLP proteins a protein of 240 kDa is described (Tarro G., Oncology 1983;40:248-253). TLP are isolated from tumor tissues as described in European patent No. 0283443 as follows. Neoplastic tissue, after removal of the necrotic material and after washing in PBS (Phosphate Buffer Solution), is minced and homogenized (as for instance in a Waring Blender) with repeated coolings in ice, and the homogenate after threefold freezing and thawing is subjected to sonication, this process being characterized in that it further comprises the successive operations of:

a) subjecting the sonicated matter to a previous centrifugation at 3,000×g according to a scheme in which the sediment from a first centrifugation, after homogenization, sonication and further centrifugation at 3,000×g gives rise to a second supernatant and to a second sediment, and the latter, subjected to the same procedure, forms a third sediment, which is discarded, and a third supernatant, which is added to the first and second supernatants to form a pool;

b) centrifuging the pool of the supernatants at 100,000×g and concentrating the supernatant;

c) introducing, after activity test, the supernatant into an isoelectric focussing (IEF) column and collecting the peak fractions, checking their activity; and

d) increasing the purification degree by treatment in a chromatographic column. Preferably, the sonication is carried out in ice at 0.9 Kc (kilocycles) and the centrifugation of the pool of the supernatants is carried out at 100,000×g for 60 minutes, the full procedure being performed at 4° C. in a sterilized environment.

Advantageously, the concentration of the final supernatant occurs by filtration on sucrose and/or other filtering aids such as polyacrylamide, Sephadex etc., and isoelectric focussing is performed at a pH of 3.5-10, the final purification step through chromatography being carried out in a DEAE A (Sepharose) column.

The physical properties of the extracted material are preferably determined on a gel of IEF column and on SDS PAGE/Sephadex as regards molecular weight and glycosidic groups.

The antigenic protein products (TLP) obtained are substantially lipoglycoproteins having the following distinctive characteristics:

a) sizes between 48.1 and 61 Angstrom (on the basis of molecular weight data known);

b) presence of a lipidic group (on the basis of elution on concavaline A);

c) solubility (undeterminable hypothetically as both density and the sedimentation coefficient are unknown); and

d) isoelectric point less than 7.0 (on the basis of the elution characteristics on Sephadex DEAE A (1 per anion) (elution with 0.2 M NaCI)).

European patent No. 649433 identifies a TLP protein obtained from pulmonary carcinoma. Italian patent application No. RM96A000496 indicates that TLP obtained from carcinomas of the urogenital system comprise peptides of a different sequence than those previously identified. The fragments of proteins from TLP can also be produced synthetically using known methods.

In 1983, a new tumor antigen of 240 kDa was identified, extracted from the neoplastic tissue of non-small cell lung carcinoma (NSCLC) and named TLP (Tumor Liberated Particle). In Cold Spring Harbor Laboratories (N.Y.-USA), a structure analyisis of the lung carcinoma antigen was recently performed and a 100 kDa antigen was extracted. A major TLP epitope sequence then was identified, the polypeptide (a nonapeptide, CSH 275) was synthesised, and the relative antibody (CSH 419) was produced.

This antibody (CSH 419) has proved its ability, using Western blot after immunoprecipitation and immunohistochemistry (P.A.P.), to recognize the antigen sequence in the homogenate obtained from all of the neoplastic tissues (NSCLC) taken into consideration up to this point.

The peptide claimed in SEQ ID NO: 1, with others derived from the antigenic region of TLP were described and claimed in the European Patent no. 649433 corresponding to the International Application WO-A-001458.

### SUMMARY OF THE INVENTION

The present invention relates to a pharmaceutical composition containing at least one protein of a TLP (Tumor Liberated Particle) complex for therapeutic and immunogenic use. The author of the present invention has now prepared full and accurate documentation of the clinical use of TLP as an immunomodulating agent (capable of stimulating the immune responses of the host) both to combat diagnosed neoplastic pathologies (immunotherapy) and to prevent cancerous pathologies (vaccine).

On the basis of experience gained during study of experimental models, the essential requirements to enable an antigen like TLP to be used from a therapeutic point of view are the following:

1. the presence of the antigen on the surface of the tumor cells;

2. the ability to pharmacologically induce or increase expression of the antigen on the surface of the cells;

3. the ability of the antigen to stimulate lymphocytes with a specific anti-tumor activity (blastogenetic capacity and CTL (cytotoxic thymus-dependent lymphocyte) activity);

4. the presence of TLP in the serum (indicates a possible correlation between serum levels and the expression of the antigen on the cell surface, and the absence of systems (kidneys or other emunctory systems such as the skin or intestine) giving rapid clearance from circulation).

The existence of these four features is indicative of both a suitability as a therapeutic agent and as a vaccine, owing to the strong immunogenic activity demonstrated.

The object of the present invention is therefore an immunogenic composition and the use thereof as a vaccine and as a medicament in the prevention and the treatment respectively of cancer, particularly pulmonary cancer and urogenital cancer, comprising at least one protein obtained from TLP or at least an immunogenic fragment thereof.

The immunogenic fragments of TLP protein preferably contain at least one of the following amino acid sequences:

ArgThrAsnLysGluAlaSerIle (SEQ ID NO: 1; identified as Seq ID N1 in WO-A-001458)

GlySerAlaXPheThrAsn (SEQ ID NO: 2; identified as Seq ID N2 in WO-A-001458)

AsnGlnArgAsnArgAsp (SEQ ID NO: 3; identified as Seq ID N3 of WO-A-001458)

Alternatively, the immunogenic composition according to the invention includes an immunogenic fragment comprising the following amino acid sequence:

GlyProProGluValGlnAsnAlaAsn (SEQ ID NO: 4; identified as Seq ID N1 of Italian patent application RM96AO00496).

### BRIEF DESCRIPTION OF THE DRAWINGS

FIG. 1 shows the result of an experiment carried out on neoplastic cells obtained from NSCLC explants using the flow cytofluorimetry technique (Facscan-BD).

The peak on the right of the histogram, reveals the binding of the anti-TLP antibodies with the corresponding antigen on the cell surface.

FIG. 2 shows the result of the same experiment described in FIG. 1, carried out on cells from the same explants, treated with pre-immune serum as a negative control.

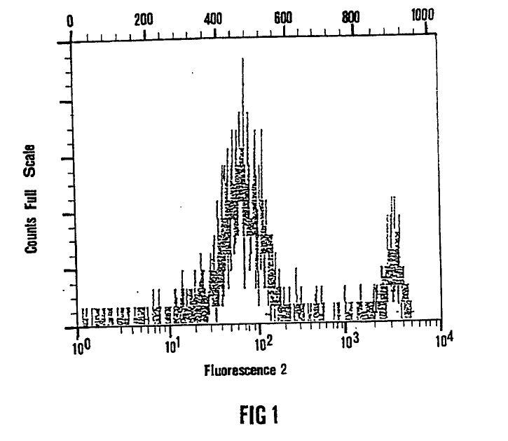

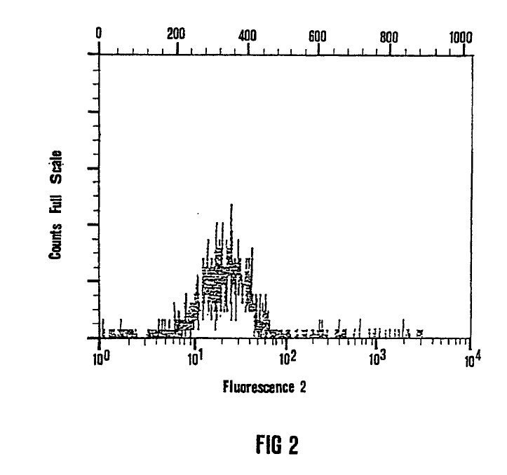

The peak shown in FIG. 1, comprising the values between 10³ and 10⁴ of the fluorescence, and revealing the bond between antigen and antibodies anti-TLP, is absent.

### EXPERIMENTAL DESCRIPTION

Presence of the Antigen TLP on the Surface of the Tumor Cells.

The antigen TLP was searched for on the surface of fresh neoplastic cells obtained from non-small cell lung carcinoma explants (NSCLC) using the flow cytofluorimetry technique (Facscan-BD).

The cells were marked by addition of monoclonal anti-TLP antibody (CSH] 419, Cold Spring Harbor Lab. N.Y. USA) in conjunction (second step) with a second goat anti-rabbit IgG-RPE.

The TLP-antiTLP binding specificity was evaluated using non-specific antisera or pre-immune serum. The cell phenotype specificity was evaluated by marking cells of stabilized tumor lines or fresh cells from neoplasias other than NSCLC with anti-TLP.

Ability to Pharmaceutically Induce or Increase Expression of the Antigen on the Surface of the Cells.

The cells were processed fresh and after preparation of primary cultures (complete RPMI 1640 culture medium with the addition of FCS 10%).

Along with variation in TLP, other possible phenotype modifications (IL-2 rec (interleukin receptors), HLA-Dr (human leukocyte antigen), CD16 (lymphocyte sub-populations), etc.) induced by treatment with single or combined agents (chemotherapy, cytokines) were also studied.

In the case of TLP, freshly isolated tumor cells were obtained from a surgical NSCLC patient. After plating for 24 h and separation of fibroblasts, cells were resuspended for immunohistochemistry and cytofluorimetric assay. Briefly, CSH 419 antiserum was labeled with PE conjugated anti-rabbit IgG and incubated with the cells. Negative controls were obtained by the use of rabbit pre-immune serum and (for immunohistochemistry also by serial dilution down to 500 fold for positive samples; no reactions were observed by staining or conjugating K562 and two melanoma cell lines. TLP was demonstrated in 75% of the NCLC lines studied.

Preliminary studies by confocal microscopy showed a cytoplasmatic and membrane localization of TLP by the tumor cells. TLP antigen expression has been shown to be enhanced or induced in vitro by chemotherapy treatment: primary NSCLC culture cells become TLP-positive after cis-platinum or etoposide treatment. The freeing of TLP in the culture supernatant was checked using the ELISA test.

Simultaneously, an experiment consisting of administration of cis-platinum and etoposide to serum-negative NSCLC patients was carried out to test the reaction on TLP production in vivo.

Ability of Ag to Stimulate Lymphocytes With Specific Anti-tumor Activity (Blastogenetic Ability and CTL Activity).

Lymphocytes obtained from peripheral blood of patients from whom the neoplastic cells were explanted (autologous lymphocytes) were marked by flow cytofluorimetry (CD4/CD25, CD8/CD25, CD56-16-3^{±}/CD25 phenotypes) before and after treatment in vitro with TLP, both alone and in association with chemotherapy and/or cytokines.

The cytotoxic activity of the autologous lymphocytes (both treated and untreated) was determined by testing release of ⁵¹Cr after 4 h, using the tumor cells obtained from explants of tumor from the same patient as the target cells.

Lymphocytes from healthy individuals and from patients suffering from other neoplastic pathologies were used (as effectors) against neoplastic cell lines (sensitive and resistant NK targets) as controls to establish the specific nature of the tumor lysis.

Presence of TLP In The Serum.

A test ELISA with analytical “sandwich” scheme, was carried out to determine the presence of TLP in the sera of NSCLC patients, in the sera of patients affected by other pathologies (neoplastic pathologies different from NSCLC; lung non-neoplastic pathologies), and in other controls.

### RESULTS

Presence of the Antigen TLP on the Surface of the Tumor Cells.

The presence of TLP on lung carcinoma cells (NSCLC) was demonstrated using the method described. As an example of a positive tumor, the data shown in FIG. 1 are reported. It is possible to note that labeling with pre-immune serum gives no signal, while labeling with anti-TLP CSH 419 distinctly demonstrates a TLP-positive population.

Ability to Pharmaceutically Induce or Increase Expression of the Antigen on the Surface of the Cells.

Two TLP-positive tumor cell populations were treated with cis-platinum and etoposide (10:g/ml) for 48 hours:

Two TLP-negative cell population became TLP-positive after etopside treatment;

One TLP-negative cell population became TLP-positive after cis-platinum treatment;

One TLP-positive cell population remained TLP positive after etoposide and cis-platinum treatment.

The results of the ELISA test used for checking the freeing of TLP in the culture supernatant are compatible with those shown in tables II-IV, reporting the presence of TLP in the sera of NSCLC patients.

The administration of the same chemotherapies in TLP serum-negative patients has produced an intense positive response for TLP, as confirmed by ELISA, after only two cycles of treatment.

Ability of Ag to Stimulate Lymphocytes With Specific Anti-tumor Activity (Blastogenetic Ability and CTL Activity).

In vitro treatment of the lymphocytes of patients suffering from NSCLC with TLP induces blastic activity, in particular against certain phenotypes: activated cells that express the high affinity receptor for IL-2 (CD3⁺/CD25⁺); NK cells (CD56⁺/CD16⁺/CD3^{±}); and activated cytotoxic cells (CD25⁺/CD8⁺).

TABLE I

| PHENOTYPES | CD25 | NK | CD25/CD8 |
|---|---|---|---|
| Untreated lymphocytes | 3.3-4.6 | 10.1-18.9 | 3.5-5.3 |
| Lymphocytes + TLP* | 5.5-16.1 | 21.4-32.2 | 8.6-11.3** |

*μg/ml
**a dose-dependent effect is observed for addition of TLP to the media and activation of the CD8⁺ cells (which can reach up to 20% of the entire culture population).

Treatment of the lymphocytes with TLP is also capable of inducing lytic activity of both types of NK (natural killer cells , target cells K562) and CTL (on cells of its own tumor). The NK activity shows the same dose dependence seen for CD8⁺/CD25 ⁺ cells and also appears to be closely correlated to the number of these cells. This observation, along with the absence of lytic activity (either spontaneous or TLP-induced) of a LAK type (lymphokine activated killer cells, targeting NK-resistant cells, Daudi) appears to indicate the specific nature of the activation.

The homologated lymphocytes, treated with TLP, have also shown themselves to be active on cells of a cerebral metastasis resulting from NSCLC.

Presence of TLP in the Serum

The results of the ELISA with analytical “sandwich” scheme has given the results shown in Tables II-V:

TABLE II
NSCLC

| HISTOLOGIC TYPE | N/POS. | % |
|---|---|---|
| Epidermoidal | 40/22 | 55 |
| Adenocarcinoma | 12/7 | 58 |
| Total | 52/29 | 56 |

TABLE III
Different neoplasias from NSCLC

| HISTOLOGICAL TYPE | N/NEG. | % |
|---|---|---|
| SCLC | 15/15 | 100 |
| Indefinite | 7/7 | 100 |
| Carcinosarcoma | 1/1 | 100 |
| Carcinoid | 1/1 | 100 |
| Pulm. Metast. from ovary | 1/0* | |
| Melanoma | 3/3 | 100 |
| Gastric Carcinoma | 2/2 | 100 |
| Total | 30/29 | 97 |

*Border line

TABLE IV
Non-neoplasia lung pathologies

| PATHOLOGY | N/NEG. | % |
|---|---|---|
| BOC | 21/18 | 86 |
| TBC | 2/1 | 50 |
| Total | 23/19 | 82 |

TABLE V
Other controls

| Cases | N/Neg. | % |
|---|---|---|
| Healthy | 11/11 | 100 |
| Pregnant | 2/1 | 50 |
| Total | 13/12 | 92 |

Moreover, the measurement of the TLP levels in NSCLC patients gives a result of 56% (see table II), which must be compared with values of 35-40% obtained using antibodies specific for “cyfra”, the only other marker proposed for lung carcinoma. Furthermore, the data indicate a binding specificity of approximately 100% for TLP versus approximately 60%-70% for “cyfra.”

In a similar manner, the synthetic polypeptide having SEQ ID NO: 1 shows excellent immunogenic abilities, with studies actually indicating a specific stimulation of the cytotoxic CD8 lymphocytes only in patents suffering from NSCLS neoplasia.

Experiments carried out in SCID mice show that a vaccinal treatment with (SEQ ID NO: 1) protects the animals from a growth of 1.8 million to 6.0 million tumor cells.

Analogous results are obtained for the other peptides claimed.

### Final Conclusion

The results of the studies carried out on TLP, the derivative peptides, and the peptide GlyProProGluValGlnAsnAlaAsn (SEQ ID NO: 4) derived from the analogous protein extracted from urogenital carcinoma, show an immunological anti-tumoral action directed against them.

In fact, the presence of TLP on the surface of the NSCLC cells, and its demonstrated capability to stimulate lymphocytes with specific anti-tumor activity (both blastogenetic ability and CTL activity), clearly demonstrate the immunogenicity of the protein. The same considerations are demonstrated to have value for the peptides represented by SEQ ID NO: 2, SEQ ID NO: 3, and SEQ ID NO: 4, also derived from the antigenic region of the TLP protein.

The value of this approach is confirmed by the fact that a relevant presence of TLP in the sera of NSCLC patients was found, indicating a strict association between the presence of tumor and the level of TLP in blood. Therefore it could be a suitable and effective approach based on the administration of TLP, or the peptides therefrom, as its immunogenically active sequences were demonstrated to have no homology to other human proteins.

The administration of TLP or peptides derived therefrom, would be more specific and effective than an approach carried out by a non-specific activation of the host's immune system, and less destructive than the administration of only chemotherapeutics.

However the administration of the chemotherapeutics could also potentiate the therapeutic effects of TLP, as they have demonstrated the capability of inducing the production of the Ag both in cell cultures and in NSCLC patients originally serum-negative for TLP.

This is a direct consequence of the results of the experiment carried out with single or combined chemotherapeutic agents. As it was previously shown in fact, the administration of etoposide and cis-platinum in tumor cell cultures was shown to stimulate TLP production at levels compatible with those registered in NSCLC serum-positive patients, and the administration of the same chemotherapies make the patients originally serum-negative (ELISA) for TLP intensively positive after two cycles of chemotherapy.

In the case of the peptide GlyProProGluValGlnAsnAlaAsn (SEQ ID NO: 4) derived from the protein analogous to TLP extracted from urogenital carcinoma, the experiments carried out demonstrate an analogous suitability as an immunotherapeutic for all forms of urogenital tumors.

All these data are indicative of both a therapeutic and a vaccinal use owing to the strong immunogenic activity demonstrated by all these molecules. This use is suitable for humans and mammals in general for proteins obtained from TLP and analogous thereto. The same is true for the peptides illustrated in the experimental texts.

The immune response in fact can be caused both to counteract tumor growth and expansion (treatment effective also in metastasis as previously shown) and to protect healthy people from developing the disease. The data obtained in SCID mice previously shown particularly support these statements.

24. A method for increasing the expression of a protein comprising an amino acid sequence of Arg Thr Asn Lys Glu Ala Ser Ile (SEQ ID NO: 1) in non-small cell lung cancer cells of a mammal, said method comprising administering to said mammal an amount of cisplatin or etopside effective in increasing the level of expression of a protein comprising an amino acid sequence of Arg Thr Asn Lys Glu Ala Ser Ile (SEQ ID NO: 1) in said non-small cell lung cancer cells of said mammal.

25. A method for increasing the expression of a protein comprising an amino acid sequence of Gly Ser Ala Xaa Phe Thr Asn (SEQ ID NO: 2) in non-small cell lung cancer cells of a mammal, said method comprising administering to said mammal an amount of cisplatin or etopside effective in increasing the level of expression of a protein comprising an amino acid sequence of Gly Ser Ala Xaa Phe Thr Asn (SEQ ID NO: 2) in said non-small cell lung cancer cells of said mammal.

26. A method for increasing the expression of a protein comprising an amino acid sequence of Asn Gln Arg Asn Arg Asp (SEQ ID NO: 3) in non-small cell lung cancer cells of a mammal, said method comprising administering to said mammal an amount of cisplatin or etopside effective in increasing the level of expression of a protein comprising an amino acid sequence of Asn Gln Arg Asn Arg Asp (SEQ ID NO: 3) in said non-small cell lung cancer cells of said mammal.

27. A method for increasing the expression of a protein comprising an amino acid sequence of Gly Pro Pro Glu Val Gln Asn Ala Asn (SEQ ID NO: 4) in urogenital cancer cells of a mammal, said method comprising administering to said mammal an amount of cisplatin or etopside effective in increasing the level of expression of a protein comprising an amino acid sequence of Gly Pro Pro Glu Val Gln Asn Ala Asn (SEQ ID NO: 4) in said urogenital cancer cells of said mammal.
